(19) United States
(12) Patent Application Publication
(10) Pub. No.: US 2019/0038415 A1
(43) Pub. Date: Feb. 7, 2019

(54) IMPLANT MOLDING SYSTEM

(71) Applicants: 3D-SIDE, Ottignies-Louvain-la-Neuve (BE); UNIVERSITÉ CATHOLIQUE DE LOUVAIN, Louvan la Neuve (BE)

(72) Inventors: Guido HEUNEN, Louvain la Neuve (BE); Michel SCLAVONS, Woluwe-Saint-Lambert (BE); Christine KINET, Woluwe-Saint-Lambert (BE); Khanh TRAN DUY, Walhain (BE); Laurent PAUL, Mellery (BE)

(21) Appl. No.: 15/760,664

(22) PCT Filed: Sep. 16, 2016
(86) PCT No.: PCT/EP2016/071927

§ 371 Date: Mar. 16, 2018

(30) Foreign Application Priority Data
Sep. 16, 2015 (EP) 15185395.9

(51) Int. Cl. A61F 2/28; A61F 2/30; B29C 33/62; B29C 33/68

(52) U.S. Cl. CPC A61F 2/2875; A61F 2/30942; B29C 33/62; A61F 2002/30962; A61F 2002/30957; A61F 2002/30948; B29C 33/68

(57) ABSTRACT

Disclosed is an implant molding system for the molding of an hardenable implant forming material including a mold obtainable from an image of a bone defect by additive manufacturing and having a surface concave along at least one axis; and a tangible unit for retaining the implant forming material in the mold during hardening and for unmolding the implant thereafter; wherein the tangible unit for retaining the implant forming material in the mold during hardening and for unmolding after hardening includes or consists of a coating to be applied onto the concave surface of the mold.

### FIELD OF INVENTION

The present invention relates to the field of molding systems for the manufacture of implants such as for example cranial implants. In particular, the present invention relates to an implant molding system for the molding of a hardenable implant forming material comprising both a mold and means for retaining the implant forming material in the mold during hardening and for unmolding the hardened implant.

### BACKGROUND OF INVENTION

In numerous situations, it may be necessary to repair cranial defects by means of cranial implants. These defects can arise from traumas, craniectomies, tumors or malformations. Cranioplasty is a neurosurgical procedure enabling to restore the protection of the brain and to improve esthetic appearance. However, the accurate restoration of the missing part is particularly challenging.

Three materials are currently available to repair bone defects: autografts, allografts and biomaterials. Autografts and allografts are naturally limited (autograft) or raises biocompatibility issues (allograft). Biomaterials may be used during surgery to manufacture implants in situ: for instance some surgeons use a glove to directly shape the bone cement onto a cranial defect. However, said method is known as being time-consuming and depends on the ability of the surgeon.

The development of computer assisted design and rapid prototyping technology increases significantly the possibility to preoperatively manufacture precise and adapted implants from biomaterials and cranial scan data. However, the scan data may not be fully reliable due to patient movement during the imaging or could become irrelevant if the surgeon decides to enlarge the bone defect during the surgery (e.g. in the case of the spread of a bone tumor). Therefore preoperatively-made implants from cranial scan data could be inappropriate to restore full protection of the brain. In order to ensure protection of the brain, optimal stress distribution along the periphery of the implant as well as the esthetic appearance; the curvature of the skull of each patient must be respected. The outer surface of the implant should be flush with the outer surface of the skull along the periphery of the implant. According to the applicant, meeting said feature with a preoperatively-made implant is one of the issue faced at each surgery.

It is thus an aspect of the present invention to provide an implant molding system for the manufacture of an implant restoring the protection of the brain while ensuring a continuous outer curvature at the boundary between the skull of the patient and the implant.

To that end, the implant molding system for the molding of an hardenable implant forming material comprises:
a mold comprising a bottom plate; and
means for retaining the implant forming material in the mold during hardening and for unmolding the implant after hardening.

The means for retaining the implant forming material in the mold during hardening and for unmolding the implant after hardening ensure unmolding of the implant while also avoiding contamination of the implant forming material by the material of the mold. As the mold ensures the correct curvature of the outer surface of the implant, due to the bottom plate, without limiting the lateral sizes of the implant, said mold may be generated using imaging such as cranial scan data of a patient having a bone defect; thereby avoiding the drawbacks of the prior art.

### SUMMARY

The present invention relates to an implant molding system for the molding of an hardenable implant forming material comprising: a mold obtainable from an image of a bone defect by additive manufacturing and having a surface concave along at least one axis; and tangible means for retaining the implant forming material, for instance against gravity, in the mold during hardening and for unmolding the implant after hardening; wherein said tangible means for retaining the implant forming material in the mold during hardening and for unmolding after hardening comprises or consists of a coating to be applied onto the concave surface of the mold wherein said coating is made of a biocompatible material and said coating has a phase transition temperature ranging from 40 to 100° C.

According to one embodiment, the coating is selected from homopolymer or copolymer of poly(isoprene), poly(chloroprene), poly(vinyl alcohol), poly(acrylonitrile), poly(butadiene), poly(vinyl chloride), poly(ethylene), poly(isobutylene), poly(ethylene glycol), poly(lactic acid) or any mixtures thereof.

According to one embodiment, the coating is selected from vaseline or wax or any mixtures thereof. According to one embodiment, the coating is vaseline. According to one embodiment, the coating is Horsley's wax.

According to one embodiment, the coating is selected from homopolymer or copolymer of poly(isoprene), poly(chloroprene), poly(vinyl alcohol), poly(acrylonitrile), poly(butadiene), poly(vinyl chloride), poly(ethylene), poly(isobutylene), poly(ethylene glycol), poly(lactic acid); or vaseline or wax or any mixtures thereof.

According to one embodiment, the coating forms a solid layer at a temperature ranging from 0 to 40° C., preferably from 25 to 40° C.

According to one embodiment, the coating is applied onto the concave surface of the mold in solid form. According to one embodiment, the coating further comprises a tab extending outside of the mold enabling peeling said coating off the mold. According to one embodiment, the coating is selected from poly(acrylonitrile-co-butadiene).

According to another embodiment, the coating is applied onto the concave surface of the mold in liquid form. According to one embodiment, the coating exhibits a melting temperature above T1 and below T2, wherein T1 is ranging from 0 to 40° C., preferably from 25 to 40° C. and wherein T2 is ranging from ranging from 45 to 100° C., preferably from 70 to 90° C. According to one embodiment, the coating is selected from homopolymer or copolymer of poly(ethylene glycol). According to one embodiment, the coating is selected from a crystalline, semicrystalline or amorphous material; preferably, the coating is selected from material having a glass temperature lower than the reaction temperature.

According to one embodiment, the implant molding system according to the invention further comprises a molding ring having a negative surface profile relative to the mold, an inner edge and a predefined thickness along the edge.

According to one embodiment, the implant molding system further comprises a forming tool having a negative surface profile relative to the mold, such that joining the forming tool with the mold, and optionally with the molding ring, forms a cavity.

According to one embodiment, the implant molding system further comprises a counter mold comprising a first and a second counter mold portion, each comprising an inner part and an outer part, wherein the counter mold and the mold form, when mated together, a cavity corresponding to the image of the bone defect.

According to one embodiment, the surface concave along at least one axis conforms to an arc with a radius ranging from 2 to 20 centimeters, from 4.5 to 9.5 centimeters, or substantially equal to 7 centimeters.

According to one embodiment, the mold and/or the molding ring and/or the forming tool and/or the counter mold comprises a material selected from ceramics, metals, metal alloys, glasses, polymers, bone or portland like cement, gypsum, sand or a mixture thereof.

According to one embodiment, the implant molding system according to the invention further comprises an implant forming composition comprising a liquid phase comprising at least one liquid monomer, and at least one polymerization inhibitor; and a solid phase comprising at least one polymer, at least one initiator; optionally at least one activator, and optionally at least one radio-opaque filler.

### Definitions

In the present invention, the following terms have the following meanings:
“Additive manufacturing” refers to a manufacturing process for shaping objects by successive addition of material.
“Bone defect” refers to a part of a bone of a subject that will not heal without intervention or to the part of a bone removed during a surgery having a size ranging from 5 to 400 cm², preferably from 30 to 150 cm².
“Coating” refers to a covering that is applied on a surface and forms a layer of solid or viscous material.
“Harden” or “hardening” refers to the action to make or to become hard or harder. According to the present invention, said action may be implemented by chemical or physical means; preferably by chemical means. According to the present invention, “harden” or “hardening” also includes when said action is implemented by using a cross-linking agent (“curing”).
“Implant forming material” refers to any material that may be formed by molding to generate a surgical prosthesis for a defect, preferably said material is hardenable.
“Mold” refers to a curved plate having at least one predefined curvature either concave or convex, preferably predefined by the specific curvature of the outer surface of the bone of a patient with a bone defect.
“Monomer” refers to compound bearing at least one function allowing the polymerization of said compound by a radical or a condensation reaction.
“Phase transition temperature” refers to the temperature needed for the material to pass from a solid or viscous phase to a liquid phase.
“Reaction temperature” refers to the highest temperature reached during hardening of the implant forming material.
“Subject” or “patient” refers to an animal, preferably a mammal, more preferably a human, in need of an implant.
“Substantially” and “about” refer, when used in conjunction with a numerical value, to the variation above or below 10% of said value, preferably above or below 5% of said value.
“Viscosity” refers to the uniform flow resistance (without turbulence) occurring in the mass of a material. In the case of a polymer solution, said viscosity depends on the temperature and the concentration of said polymer solution and its molecular weight and structure.
“Viscous” refers herein to a compound having a viscosity value higher than 80 mPa·s at 20° C. The viscosity may be measured by any appropriate viscometer, method or apparatus known by the skilled artisan, such as but not limited to, viscometer of Oswald or Ubbelohde, falling ball viscometer, rotative or Couette viscometer.

### DETAILED DESCRIPTION

This invention relates to an implant molding system for the molding of an hardenable implant forming material comprising:
a mold 2 having at least one predefined curvature;
means for retaining the implant forming material in the mold 2 during hardening and for unmolding the hardened implant, especially tangible means; and
optionally a molding ring 3.

According to one embodiment, the implant molding system is used for manufacturing implants in human and veterinary medicine. According to a preferred embodiment, the implant molding system is used for manufacturing implants such as cranial implants, preferably neurocranial implant. According to a preferred embodiment, the implant molding system is used for manufacturing implants to repair bone defect as shown in FIG. 1. However, the present invention is not limited to cranial implant and one skilled in the art could use the implant molding system of the invention to manufacture other implants such as for instance zygomatic implants, maxillofacial implants, chin implants or pelvic implants.

According to a first embodiment, the mold 2 comprises a surface concave along at least one axis. According to one embodiment, the mold 2 comprises a partly concave surface along at least one axis. According to another embodiment, the mold 2 comprises a surface convex along at least one axis. According to one embodiment, the mold 2 comprises a partly convex surface along at least one axis. According to one embodiment, the surface of the mold 2 is concave and substantially conforms to an arc with a radius ranging from 2 to 20 centimeters, preferably from 4.5 to 9.5 centimeters, or substantially equal to 7 centimeters.

According to one embodiment, the mold 2 comprises an inner face 22 and an outer face 21 as shown in FIGS. 2A and 2B. The said inner face 22 is designed to be in contact with the implant forming material and the said outer face 21 is oriented towards outward. According to one embodiment, the mold 2 comprises a handle 23 on its outer face 21.

According to one embodiment, the mold 2 has the shape of the bone defect 1 to be replaced or restored. According to one embodiment, the mold 2 is patient-specific. According to one embodiment, the mold 2, which has the shape of the bone defect 1 to be replaced or restored, is designed via medical imaging. It may be manufactured by additive manufacturing such as 3D printing.

According to one embodiment, a digital imaging system, such as for example a computer tomography (CT) or Magnetic resonance imaging (MRI) is used to obtain an imaging data pertaining to a patient's anatomy. The imaging data is then imported into a standard image format by use of a suitable software, such as for example Mimics® (Materialise). This enables the creation of a 3D model of the patient anatomy. Having obtained a 3D model of the patient anatomy, a model of the mold 2 having a surface corresponding to the bone defect 1 including size and curvature thereof is produced. From the model, the mold 2 is processed and manufactured by any technique known by one skilled in the art such as additive manufacturing (e.g. binder jetting, material extrusion, material jetting, powder bed fusion, sheet lamination, selective laser sintering, sheet metal forming . . . ). Thus according to one embodiment, the mold 2 has a curvature ensuring continuity and/or tangency at the boundary between the bone of the patient, for instance a skull, and the implant. According to one embodiment, the mold 2 has a curvature ensuring flush with the boundary between the bone of the patient, for instance a skull, and the implant.

According to one embodiment, the mold is obtained by adapting a standard plate or a standard mold to a desire implant shape, for instance by bending and/or forming.

According to an alternative embodiment, the mold 2 is generated from bone models having standard curvatures. For instance, the mold 2 is generated from a skull model having standard curvature. The curvature of the skull typically ranges from 2 centimeters to 20 centimeters, preferably from 4.5 to 9.5 centimeters or is approximately equal to 7 centimeters. The standard mold may then be adapted during surgery to the patient anatomy to be perfectly flush.

According to one embodiment, the mold 2 is manufactured preoperatively. According to one embodiment, the mold 2 is manufactured per-operatively.

According to one embodiment, the mold 2 has a surface larger than the size of the bone defect to be repaired or restored. Preferably, the mold 2 has a surface larger than the size of the bone defect to be replaced such that the surgeon may use the mold 2 even if he decides during the surgery to enlarge the bone defect for medical reason. According to one embodiment, the edge of the mold 2 corresponds to the edge of the bone defect to be replaced or restored plus 20%, 10%, 5% or 3%.

According to one embodiment, the implant molding system further comprises a molding ring 3 cooperating with the mold 2 as shown in FIGS. 3 and 4A. In the sense of the present invention cooperating refers to the fact that the molding ring 3 has a negative surface profile relative to the mold 2 such that joining the molding ring 3 with the mold 2 enable to guide the thickness and the edge of the implant.

According to one embodiment, the mold 2 has a surface larger than the bone-defect thereby enabling the molding ring 3 to be positioned on the concave surface of the mold 2 and to define the thickness and the edge of the implant as shown in FIG. 4B.

According to one embodiment, the molding ring 3 has a thickness substantially equal to the thickness of the bone defect 1 at its periphery. According to one embodiment, the inner contour of the molding ring 3 corresponds to the outer edge of the defect to be repaired or restored. According to one embodiment, when the molding ring 3 is placed in a concave mold 2, the molding ring 3 cooperates with the mold 2 and forms a rim oriented towards the concavity of the mold 2. According to one embodiment, when the implant forming material is pressed and shaped in the mold 2, the molding ring 3 ensures correct shaping of the implant forming material (i.e. with the right thickness and outer size) as shown in FIGS. 5A and 5B.

According to one embodiment, the molding ring 3 is designed via medical imaging. It may be manufactured by additive manufacturing. According to one embodiment, the molding ring 3 is patient-specific. According to one embodiment, the molding ring 3 is manufactured preoperatively. According to one embodiment, the molding ring 3 is manufactured per-operatively.

According to one embodiment, the implant is made by an implant molding system comprising a mold 2, the tangible means for retaining the implant forming material in the mold 2 during hardening and for unmolding the hardened implant and a forming tool. According to one embodiment, the implant is made by an implant molding system comprising a mold 2, the tangible means for retaining the implant forming material in the mold 2 during hardening and for unmolding the hardened implant and a counter mold 4, 5.

According to one embodiment, the implant molding system further comprises a forming tool having a negative surface profile relative to the mold 2, such that joining the forming tool with the mold 2, and optionally with the molding ring 3, forms a cavity. Said cavity corresponds substantially to the shape of the defect 1 to be repaired.

According to one embodiment, the forming tool is used temporarily to shape the implant forming material.

According to one embodiment, the forming tool ring is designed via medical imaging. It may be manufactured by 3D printing technology. According to one embodiment, the forming tool 3 is patient-specific. According to one embodiment, the forming tool is manufactured preoperatively. According to one embodiment, the forming tool is manufactured per-operatively.

According to one embodiment, the counter mold 4, 5 is used temporarily to shape the implant forming material, especially its thickness.

According to one embodiment a counter mold 4 is used in the first steps of the implant molding process to facilitate the filing by the implant forming material in liquid phase within the mold 2. As the implant forming material in liquid phase paste quickly, assuring a better filling by avoiding air bubbles is necessary and is realized by the use of a divided counter mold 4. According to one embodiment, the counter mold 4 is divided into two portions to permit, during assembly of the two parts filled with the implant forming material in liquid phase overflowing, producing enough pressure to ensure a good contact for a better implant quality.

According to one embodiment, the implant molding system further comprises a counter mold 4. According to one embodiment, the implant molding system does not comprise a molding ring 3. According to one embodiment, the counter mold 4 is a divisible counter mold 4 including a first counter mold portion 41 and a second counter mold portion 42. According to one embodiment and represented in FIG. 6, each of the said counter mold portion is defined by two parts: an outer part, 412 and 422, and an inner part, 411 and 421. According to one embodiment, each part of the counter mold portion comprises outer faces, 4112, 4122, 4222 and 4212, and inner faces, 4111, and 4211, inner faces are oriented towards molding and outer faces are oriented towards outward. The said inner faces of the first 41 and second 42 counter mold portions outer parts, 412 and 422, are shaped for receiving the mold 2.

According to one embodiment, the said outer parts, 412 and 422, of the counter mold portions, 41 and 42, comprise a housing for the mold handle 23. According to one embodiment and represented in FIG. 7, the said outer parts, 412 and 422, of the counter mold portions, 41 and 42, comprise a hole when gathered for the mold handle 23 to pass through. According to one embodiment, the said outer parts, 412 and 422, of the counter mold portions, 41 and 42, don't have a hole.

According to one embodiment, the mold 2 comprises a handle 23 on its outer face 21. According to one embodiment, the mold 2 includes two mold portions to form a mold 2 with a concave shape on its inner face 22 when mated together, a first mold portion 24 and a second mold portion 25. According to one embodiment, the first mold portion 24 has a first mold portion handle 241 and the second mold portion 25 has a second mold portion handle 251. According to one embodiment, the first mold portion handle 241 and the second mold handle 251 comprise at least one fixing system to hold the two mold portions, 24 and 25, together. According to one embodiment, the first 24 and the second 25 mold portion comprise a fixing system to hold the two mold portions together.

According to one embodiment, the said inner face of the implant is placed within the subject body and the said outer face of the implant is placed on the surface of the subject body.

According to one embodiment, the inner faces of the outer parts, 412 and 422, of the first 41 and second 42 counter mold portions have a shape that interlock with the outer face 21 of the mold 2. According to one embodiment, the inner faces of the outer parts of the first 41 and second 42 counter mold portions are fixed to the outer face 21 of the mold 2 by a binding agent such as glue. According to one embodiment, the external face of implant has a shape defined by the shape of the inner face 22 of the mold 2 and the shape of the internal face of the implant is defined by the shape of the inner faces of the inner parts, 411 and 421, of the first 41 and second 42 counter mold portions, 4111 and 4211.

According to one embodiment, the outer faces of the inner parts, 411 and 421, of the first 41 and second 42 counter mold portions have a particular shape such as a planar or a concave surface. According to one embodiment, the outer faces of the inner parts, 411 and 421, of the first 41 and second 42 counter mold portions, 4112 and 4212, have a random shape.

According to one embodiment and as represented in FIGS. 8A and 8B, the inner 411 and outer 412 parts of the first counter mold portion 41 comprise at least one fixing system 413 to hold them together. According to one embodiment, the fixing system 413 renders the said inner 411 and outer 412 parts of the first counter mold portion 41 dependent. According to this embodiment, the first counter mold portion 41 comprises the first mold portion 24.

According to one embodiment, the said inner 421 and outer 422 of the second counter mold portion 42 comprise at least one fixing system 423 to hold them together. According to one embodiment, the fixing system 423 renders the said inner 421 and outer 422 parts of the second counter mold portion 42 dependent. According to this embodiment, the second counter mold portion 42 comprises the second mold portion 25.

According to one embodiment, the inner parts, 411 and 421, of the first 41 and second 42 counter mold portions comprise at least one fixing system 43 represented in FIGS. 9A and 9B. According to one embodiment, the fixing system 43 between the two inner parts, 411 and 421, of the first 41 and second 42 counter mold portions can be defined as interlocking elements.

According to one embodiment, the two mold portions, 24 and 25, and the four parts, 411, 412, 421 and 422, of the counter mold 4 form a cavity localized between the inner face 22 of the mold 2 and the inner faces, 4111 and 4211, of the inner parts, 411 and 421, of the counter mold 4.

According to another embodiment, as depicted in FIGS. 10A, 10B, 10C and 10D, the counter mold 5 comprises a first portion 51 and a second portion 52. According to one embodiment a counter mold 5 is used in the first steps of the implant molding process to facilitate the filing by the implant forming material of the mold 2. According to one embodiment, the counter mold 5 and the mold 2 form, when mated together, a cavity corresponding to the image of the bone defect.

According to one embodiment, the first portion 51 comprises a hole for the mold handle 23 to pass through. According to one embodiment, the first portion 51 comprises a part exhibiting a predefined curvature corresponding to the curvature of the mold 2 and the second portion 52 comprises a part exhibiting a negative surface profile relative to the mold 2. According to one embodiment, the first portion 51 and the second portion 52 are hinged together, preferably on one side by a revolute joint, such that when the implant forming material is deposited on the mold located within the counter mold 5, and when the first portion and the second portion 51, 52 are mated together, the implant forming material is pressed homogeneously on the mold 2.

According to one embodiment, the counter mold 4, 5 is designed via medical imaging. It may be manufactured by 3D printing technology. According to one embodiment, the counter mold 4, 5 is patient-specific. According to one embodiment, the counter mold 4, 5 is manufactured preoperatively. According to one embodiment, the counter mold 4, 5 is manufactured per-operatively.

According to one embodiment, the mold 2 and/or the molding ring 3 and/or the forming tool and/or the counter mold 4, 5 is made of a biocompatible material. According to one embodiment, the mold 2 and/or the molding ring 3 and/or the forming tool and/or the counter mold 4,5 is made of ceramics, metals, metal alloys, glasses, polymers, bone or Portland like cement, gypsum, sand, stem cells or mixture thereof. According to one embodiment, the material of the mold 2 and/or the molding ring 3 and/or the forming tool and/or the counter mold 4, 5 is selected from any materials suitable for additive manufacturing known by one skilled in the art. According to one embodiment, the mold 2 is a composite element comprising reinforcing element such as for instance metals, glasses or carbon fibers.

In one embodiment, the material of the mold 2 and/or the molding ring 3 and/or the forming tool and/or the counter mold 4,5 is selected from the group of polymer; preferably from the group of homopolymer or copolymer comprising polyamide (PA), poly(lactic acid) (PLA), poly(styrene), poly(butadiene) or poly(acrylonitrile); more preferable the mold material is selected from aliphatic polyamide such as but not limited to polycaprolactame (PA6), polylauroamide (PA12), polyundecanamide (PA11), polytetramethylene adipamide (PA4.6), polyhexylmethylene adipamide (PA6.6), polyhexylmethylene nonanediamide (PA6.9), polyhexylmethylene decanamide (PA6.10), polyhexylmethylene dodecanamide (PA6.12), polydecamethylene decanamide (PA10.10), polydecamethylene dodecanamide (PA10.12); semi-aromatic polyamide or polyphtalimide such as but not limited to polyhexylmethylene isophtalamide (PA 6.I), polyhexylmethylene terephtalamide (PA 6.T), polymetaxylene adipamide (PA mXD.6); aromatic polyamide or aramides such as but not limited to polymetaphenylene isophtalamide (PA MPD.I) and polyparaphenylene terephtalamide (PA PPD.T). In one embodiment, the mold 2 and/or the molding ring 3 and/or the forming tool and/or the counter mold 4,5 is made of High Impact Poly(styrene) (HIPS). In one embodiment, the mold 2 and/or the molding ring 3 and/or the forming tool and/or the counter mold 4, 5 is made of a poly(acrylonitrile-co-butadiene-co-styrene) (ABS). In one embodiment, the mold 2 and/or the molding ring 3 and/or the forming tool and/or the counter mold 4, 5 is made of poly(etheretherketone) (PEEK). In one embodiment, the mold 2 and/or the molding ring 3 and/or the forming tool and/or the counter mold 4,5 is made of polyamide, such as PA12 or PA2200 commercialized by E-MANUFACTURING SOLUTIONS. In one embodiment, the mold 2 and/or the molding ring 3 and/or the forming tool and/or the counter mold 4, 5 is made of poly(ethylene-co-butylene terephthalate).

In one embodiment, the material of the mold 2 and/or the molding ring 3 and/or the forming tool and/or the counter mold 4, 5 does not deform during hardening of the implant forming material. In one embodiment, the material of the mold 2 and/or the molding ring 3 and/or the forming tool and/or the counter mold 4, 5 is selected from the group of polymer having a glass-transition temperature (T_{g}) equal or higher than the room temperature.

In one embodiment, the material of the mold 2 and/or the molding ring 3 and/or the forming tool and/or the counter mold 4, 5 is selected from the group of polymer having a melting temperature (Tₘ) higher than the reaction temperature. In one embodiment, the material of the mold 2 and/or the molding ring 3 and/or the forming tool and/or the counter mold 4, 5 is selected from the group of polymer having a glass temperature (T_{g}) lower than the reaction temperature. In one embodiment, the material of the mold 2 and/or the molding ring 3 and/or the forming tool and/or the counter mold 4, 5 is selected from the group of polymer having a melting temperature (Tₘ) higher than 45° C., preferably higher than 70° C., more preferably higher than 90° C., even more preferably higher than 110° C. In one embodiment, the melting temperature (Tₘ) is at least 50° C. higher than the reaction temperature of the implant.

According to one embodiment, the material of the mold 2 and/or the molding ring 3 and/or the forming tool and/or the counter mold 4,5 resists (i.e. does not deform if exposed) to temperatures higher than room temperature; preferably higher than 40° C.; more preferably higher than 90° C. In one embodiment, the material of the mold 2 and/or the molding ring 3 and/or the forming tool resists to temperature around 90° C. In one embodiment, the material of the mold 2 and/or the molding ring 3 and/or the forming tool and/or the counter mold 4,5 resists to temperature about 135° C. According to one embodiment, said temperature is achieved by an external source of heat.

According to one embodiment, the material of the forming tool is selected from homopolymer or copolymer of polysiloxan (also called silicone).

The implant molding system of the present invention also comprises means for retaining the implant forming material in the mold 2 during hardening and for unmolding the hardened implant thereafter. According to one embodiment, said means is a coating.

According to one embodiment, the mold 2 of the invention is larger than the bone defect 1 of a patient such that the mold rests on the existing bone around the defect 1 during testing by the surgeon; thereby providing the surgeon the ability to modify the implant geometry depending on the size of the opening performed to repair or restore the bone defect 1. In order to ensure that when testing the implant in situ, the curvature is conserved, the surgeon holds the implant within the mold. The adhesion between the implant forming material and the mold 2 must thus be sufficient in order to avoid deformation or loss of the implant during testing and should also enable unmolding without deformation after hardening. When unmolding is required after hardening of the implant forming material, the adhesion between the implant molding material and the mold 2 should also be sufficiently weak to ensure unmolding without structural deformation and cross-contamination. To that end, the implant molding system of the invention comprises means for retaining the implant forming material in the mold 2 during hardening and for unmolding the hardened implant. Said means also avoid contamination of the implant forming material by the material of the mold 2.

According to one embodiment, the means for ensuring adhesion between the implant forming material and the mold 2 during hardening and for unmolding the hardened implant is a coating.

According to one embodiment, said coating allows: 1) ensuring adhesion between the hardening implant material and the mold 2 during hardening; and 2) ensuring easy unmolding after hardening of the implant while avoiding contamination of said implant by the material of the mold 2.

According to on embodiment, said adhesion is sufficient to retain the implant forming material in the mold even if the concave surface of the mold 2 is directed downwards during 2, 3, 4, 5 or 10 minutes.

Consequently, said coating retains the implant forming material against gravity and potential forces during implant forming material application and during adaptation, e.g. on the patient.

According to one embodiment, in a first state said coating ensures that the implant forming material stays on the mold and in a second state, said coating enables easy unmolding of the implant from the mold.

According to one embodiment, the ability of implant unmolding depends on the reduction of the physical adhesion between the implant and the mold 2 material.

According to one embodiment, once applied on the surface of the mold 2, at room temperature or at a temperature ranging from 0 to 30° C., preferably from 15 to 25° C., the coating forms a solid or viscous layer.

According to one embodiment, the coating is selected from vaseline, or Horsley's wax or homopolymer or copolymer of poly(isoprene), poly(chloroprene), poly(vinyl alcohol), poly(acrylonitrile), poly(butadiene), poly(vinyl chloride), poly(ethylene), poly(isobutylene), poly(ethylene glycol), poly(lactic acid) or any mixtures thereof.

According to one embodiment, the coating is applied on the surface of the mold 2 in a liquid or viscous form.

According to one embodiment, the coating is a biocompatible compound. According to one embodiment, the coating is a bioresorbable compound.

In one embodiment, the coating is selected from biocompatible polymer selected from functionalized or unfunctionalized poly(ethylene glycol), preferably PEG 1000 or PEG 1500. PEG 1000 and PEG 1500 have the advantage of being sticky in solid form. In such a manner, it ensure retention between the mold and the implant forming material in the mold 2 during hardening due to physical adhesion between the implant forming material and the PEG. When the PEG is liquid, it does not ensure retention and the implant can be easily unmold from the mold. In one embodiment, the coating is a homopolymer or copolymer comprising poly(ethylene glycol) moiety. In one embodiment, the coating is a biocompatible lubricant such as but not limited to petroleum jelly (vaseline), natural or synthetic oil, waxes such as Horsley's wax or bone wax, macroproteins such as gelatin, glucid such as sugar or starch. In one embodiment, the coating is selected from biocompatible polymer selected from poly(lactic acid).

In one embodiment, the coating is selected from Horsley's wax or vaseline or homopolymer or copolymer of poly(ethylene glycol) or any mixtures thereof.

In one embodiment, the coating is selected from a biocompatible polymer having a molar weight ranging from 130 g/mol to 1 000 000 g/mol; preferably ranging from 500 g/mol to 800 000 g/mol; more preferably ranging from 800 to 10 000 g/mol. In one embodiment, the coating is a biocompatible polymer having a molar weight of about 1 000 g/mol.

According to one embodiment, in order to apply the coating on the mold 2, the coating is solubilized with at least one solvent such as for instance water, alcohol, acetone or chlorinated solvents. According to an embodiment, a diluted solution of the coating is applied on the mold 2. In one embodiment, the solution comprising a coating may further comprise a dye and/or pigment and/or an antibiotic. In one embodiment, the solvent is human-safe. In one embodiment, the solvent may be easily evaporated after deposition on the mold 2.

According to one embodiment, the coating is melt before application. According to one embodiment a molten coating is applied on the mold 2 and then cooled until it forms a solid layer, before application of the implant forming material.

According to one embodiment, the coating also comprises a dye.

According to one embodiment, the coating is selected from the group of polymer having a melting temperature (T_{m coating}) higher than T₁ and lower than T₂, wherein T₁ is the room temperature and T₂ is the reaction temperature of the implant manufacture.

According to one embodiment, the coating is selected from the group of polymer having a melting temperature (T_{m coating}) higher than T₁ and lower than T₂, wherein T₁ is ranging from 0 to 40° C., preferably from 25 to 40° C., more preferably about 40° C. and T₂ is ranging from 45 to 100° C., preferably from 70 to 90° C., more preferably about 60° C.

In one embodiment, the coating is selected from the group of polymer having a phase transition at a temperature higher than T₁ and lower than T₂, wherein T₁ is ranging from 0 to 40° C., preferably from 25 to 40° C., more preferably about 40° C. and T₂ is ranging from 45 to 100° C., preferably from 70 to 90° C., more preferably about 60° C.

In one embodiment, the coating is selected from the group of polymer having a phase transition temperature ranging from 40 to 100° C., from 40 to 90° C., from 40 to 80° C., from 40 to 70° C., from 40 to 65° C. or from 45 to 60° C. In said embodiment, the phase transition temperature does not refer to the glass-transition temperature.

In one embodiment, the coating is selected from the group of polymer having a melting point from 45 to 60° C. from 40 to 100° C., from 40 to 90° C., from 40 to 80° C., from 40 to 70° C., from 40 to 65° C. or from 45 to 60° C.

In one embodiment, the coating is viscous or solid at ambient temperature and is liquid at the reaction temperature of the implant manufacture.

According to one embodiment, the coating in liquid or viscous form is deposited on the mold by any means known by one skilled in the art such as for instance thermoforming, spray-coating, dip-coating, painting, spin-coating or calendering.

According to one embodiment, the coating ensures retention of the implant forming material in the mold 2 during hardening due to physical adhesion between the implant forming material and the coating. According to one embodiment, the coating ensures unmolding of the hardened implant due to the temperature of the exothermic reaction of hardening of the implant forming material which exceed the melting temperature of the coating. According to an alternative embodiment, the coating ensures unmolding of the hardened implant due to the heating of the coating by means of an external heat source at a temperature above the melting temperature of the coating.

According to one embodiment, the coating is applied on the surface of the mold 2 in a solid or viscous form.

According to one embodiment, the coating ensures retention of the implant forming material in the mold 2. After the coating turns into a liquid form, it does not ensure retention anymore and ensures unmolding of the hardened implant.

According to one embodiment, the coating is applied in the form of a solid film. According to one embodiment, the solid film is made of biocompatible polymer. In one embodiment, the solid film is a polymer selected from homopolymer or copolymers of poly(isoprene), poly(chloroprene), poly(vinyl alcohol), poly(acrylonitrile), poly(butadiene), poly(vinyl chloride), poly(ethylene), poly(isobutylene); preferably is a poly(acrylonitrile-co-butadiene).

According to an embodiment, the solid film is selected from pressure-sensitive tape or nitrile butadiene rubber.

According to one embodiment, once applied on the mold 2, the solid film comprises a tab extending outside of the mold 2 enabling peeling said coating off the mold 2. According to one embodiment, once applied on the mold 2, the solid film comprises a tab extending outside of the mold 2 enabling peeling said coating and the implant off the mold 2.

According to one embodiment, the solid film ensures retention of the implant forming material in the mold 2 during hardening due to physical adhesion between the implant forming material and the solid film. According to one embodiment, the solid film ensures unmolding of the hardened implant by peeling said film off the mold 2.

According to one embodiment, the implant forming material is selected from any implant forming material known by one skilled in the art such as for instance Palacos® or Palamed® commercialized by Heraeus.

According to one embodiment, the implant forming material is selected from biocompatible materials suitable for class IIb or class III medical devices. In one embodiment, the implant comprises a cross-linked polymer. In one embodiment, the implant does not comprise any cross-linked polymer.

According to one embodiment, the implant forming material mainly comprises a material having a glass-transition temperature higher than 40° C.; preferably higher than 90° C. In one embodiment, the implant mainly comprises a material having a glass-transition temperature about 110° C.

According to one embodiment, the implant forming material of the implant molding system comprises a composition comprising the mixture of a liquid phase comprising:
at least one liquid monomer; and
at least one polymerization inhibitor;
   and a solid phase comprising:
at least one polymer;
at least one initiator;
optionally at least one activator; and
optionally at least one radio-opaque filler.

According to one embodiment, the liquid monomer is selected from the group of acrylates, methacrylates, styrenics, acrylamides, amides, urethanes, amino acids such as but not limited to lactic acid; preferably selected from the group of acrylates, methacrylates or combination thereof; more preferably the monomer is methyl methacrylate. In one embodiment, the liquid monomer is methyl methacrylate.

According to one embodiment, the polymerization inhibitor is hydroquinone.

According to one embodiment, the liquid phase further comprises at least one dye and/or pigment selected from at least one biocompatible dye and/or pigment; more preferably from food additives. In one embodiment, the liquid phase further comprises additive copper complexes of chlorophylls (E141).

According to one embodiment, the polymer powder comprises homopolymer or copolymer obtained by the polymerization of at least one monomer selected from the group of acrylates, methacrylates, styrenics, acrylamides, amides, urethanes, amino acids such as but not limited to lactic acid; preferably selected from the group of acrylates, methacrylates or combination thereof; more preferably the polymer powder is a copolymer of methyl methacrylate and methyl acrylate.

In one embodiment the polymer powder comprises random, block, linear, dendritic, branched, cross-linked polymer or combination thereof.

According to one embodiment, the initiator is selected from a photoiniator, a radical initiator or a redox initiator; preferably from a diazoinitiator, such as azobisisobutyronitrile (AIBN), 2,2′-azobis(2-methylpropionamidine); a peroxide, such as acyl peroxides, acetyl peroxides, benzoyl peroxides, alkyl peroxides, hydroperoxides, acyl alkylsulfonyl peroxides, dialkyl peroxydicarbonates, diperoxyketals, ketone peroxides; a perester; an azo; a disulfide; a tetrazene; a persulfate compounds.

In one embodiment, the initiator is benzoyl peroxide.

According to one embodiment, the photoinitiator is selected from 2-tert-butylanthraquinone; camphorquinone; diphenyl(2,4,6-trimethylbenzoyl)phosphine oxide; 9,10-phenanthrenequinone; phenylbis(2,4,6-trimethylbenzoyl)phosphine oxide; 2-benzyl-2-(dimethylamino)-4′-morpholinobutyrophenone; 3,6-bis(2-methyl-2-morpholinopropionyl)-9-octylcarbazole; 4′-tert-butyl-2′,6′-dimethylacetophenone; 2,2-diethoxyacetophenone; 4′-ethoxyacetophenone; 3′-hydroxyacetophenone; 4′-hydroxyacetophenone; 1-hydroxycyclohexylphenylcetone; 2-hydroxy-4′-(2-hydroxyethoxy)-2-methylpropiophenone; 2-hydroxy-2-methylpropiophenone; 2-methyl-4′-(methylthio)-2-morpholinopropiophenone; 4′-phenoxyacetophenone; benzoin; benzoinethyl ether benzoinmethyl ether; 4,4′-dimethylbenzoin, 4,4′-dimethylbenzil; benzophenone; benzoylbiphenyl; 4,4′-bis(diethylamino)benzophenone; 4,4′-dihydroxybenzophenone; 3,4-dimethylbenzophenone; 3-hydroxybenzophenone; 4-hydroxybenzophenone; 2-methylbenzophenone; 3-methylbenzophenone; 4-methylbenzophenone; methylbenzoylformate; Michler ketone; 1-chloro-4-propoxy-9H-thioxanthen-9-one; 2-chlorothioxanthen-9-one; 2,4-diethyl-9H-thioxanthen-9-one; isopropyl-9H-thioxanthen-9-one; 10-methylphenothiazin; thioxanthen-9-one; preferably from 2,4-dihydroxybenzophenone, ethyl 4-dimethylaminobenzoate and camphorquinone.

According to one embodiment, the solid phase comprises at least one activator such as for instance N,N-dimethyl-p-toluidine.

According to one embodiment, the solid phase comprises at least one radio-opaque filler such as for instance zirconium dioxide.

According to one embodiment, the solid phase further comprises at least one dye and/or pigment selected from at least one biocompatible dye and/or pigment; more preferably the at least one dye and/or pigment is selected from food additives. In one embodiment, the solid phase further comprises additive copper complexes of chlorophylls (E141).

According to one embodiment, the solid phase further comprises at least one antibiotic such as for instance gentamicin.

According to one embodiment, the composition is hardened by the mixture between the solid phase and the liquid phase. In one embodiment, the hardener is the initiator.

In one embodiment, the mixture between the solid phase and the liquid phase is carried out in the open air. In one embodiment, the mixture between the solid phase and the liquid phase is carried out under inert gas.

In one embodiment, the mixture between the solid phase and the liquid phase leads to a homogeneous paste. In one embodiment, the mixture between the solid phase and the liquid phase leads to a sticky paste. In one embodiment, the mixture between the solid phase and the liquid phase leads to a paste with good handling properties.

The invention also relates to a process for manufacturing the coated implant molding system comprising the following steps:
providing:
   a mold 2 having a surface concave along at least one axis obtained by additive manufacturing from medical imaging;
   optionally, a molding ring 3 obtained by additive manufacturing from medical imaging;
   optionally, a forming tool obtained by additive manufacturing from medical imaging; and
   means for retaining the implant forming material in the mold 2 during hardening and for unmolding the hardened implant; and
coating the concave surface of the mold 2 with the means for retaining the implant forming material in the mold 2 during hardening and for unmolding the hardened implant thereafter.

The invention also relates to a process for manufacturing the implant comprising the following steps:
providing:
   a mold 2 having a surface concave along at least one axis obtained by additive manufacturing from medical imaging;
   optionally, a molding ring 3 obtained by additive manufacturing from medical imaging;
   optionally, a forming tool obtained by additive manufacturing from medical imaging;
   an implant forming material comprising a composition comprising a liquid phase comprising at least one monomer and at least one polymerization inhibitor; and a solid phase comprising at least one polymer, at least one initiator, at least one activator and at least one radio-opaque filler; and
   means for retaining the implant forming material in the mold 2 during hardening and for unmolding the hardened implant; and
coating the concave surface of the mold 2 with the means for retaining the implant forming material in the mold 2 during hardening and for unmolding the hardened implant thereafter;
mixing the liquid phase comprising at least one liquid monomer and at least one polymerization inhibitor; with the solid phase comprising at least one polymer, at least one initiator, at least one activator; and at least one radio-opaque filler;
shaping the implant forming material by spreading out the implant forming material onto the concave surface of the mold 2; optionally by means of the molding ring 3 and/or the forming tool;
hardening of the implant during a setting time;
unmolding the implant from the mold 2;
optionally heating the implant by means of an external heat source;
adapting the implant by making holes or the like.

According to one embodiment, in order to avoid residual monomers, post-hardening heating is achieved preferably up to 95, 90, 85, 80 or 75° C. during 5, 10, 15, 20, 30, 45 or 60 minutes.

According to one embodiment, post heating is achieved preferably up to 90° C. during 15 minutes.

According to one embodiment, the process is exothermic. According to one embodiment, if the process is not exothermic the heat source is provided by an external source.

According to one embodiment, the implant forming material is spread out in the mold 2, and optionally in the molding ring 3, and is pressed to be shaped.

According to one embodiment, the implant forming material is pressed using a forming tool having a negative surface profile relative to the mold 2, such that joining the forming tool with the mold 2, and optionally with the molding ring 3, forms a cavity having the shape and thickness of the defect.

According to one embodiment, the implant forming material may be shaped (shaping time) between 30 seconds and 15 minutes after mixing the solid phase and the liquid phase of the implant forming material, preferably between 1 and 12 minutes, more preferably between 1 and 7 minutes. According to one embodiment, in order to extend the shaping time, the liquid and solid phases of the implant forming material are cooled down before mixing.

According to one embodiment, the setting time is ranging from 5 to 30 minutes, preferably from 10 to 15 minutes.

The invention also relates to a process for manufacturing the implant comprising the following steps:
providing:
   a mold 2 having a surface concave along at least one axis obtained by additive manufacturing from medical imaging, the said mold 2 is divided in a first 24 and a second 25 mold portions;
   a counter mold 4,5 divided in two counter mold portions: the first 41 and the second 42 counter mold portions, wherein each of the portion is divided in two parts: the inner, 411 and 421, and the outer parts, 412 and 422; when the four parts are mated together the counter mold 4 comprises a cavity correspondingly shaped with the mold 2 and the desired implant;
   an implant forming material comprising a composition comprising a liquid phase comprising at least one monomer and at least one polymerization inhibitor; and a solid phase comprising at least one polymer, at least one initiator, at least one activator and at least one radio-opaque filler; and
coating the concave surface of the mold 2 with the means for retaining the implant forming material in the mold 2 during hardening and for unmolding the hardened implant thereafter;
fixing the first mold portion 24 onto the inner face of the outer part 412 of the first counter mold portion 41 optionally by means of binding agent;
assembling the inner part 411 and the outer part 412 of the first counter mold portion 41 by means of at least one fixing system, wherein the first mold portion 24 is fixed, by means of at least one fixing system;
fixing the second mold portion 25 onto the inner face of the outer part 422 of the second counter mold portion 42 optionally by means of binding agent;
assembling the inner part 421 and the outer part 422 of the second counter mold portion 42, wherein the second mold portion 25 is fixed, by means of at least one fixing system;
mixing the liquid phase comprising at least one liquid monomer and at least one polymerization inhibitor; with the solid phase comprising at least one polymer, at least one initiator, at least one activator; and at least one radio-opaque filler;
shaping the implant forming material by pouring the implant forming material into the cavity of the first 41 and of the second 42 counter mold portions;
assembling the first 41 and second 42 counter mold portions by means of at least one fixing system 43;
hardening of the implant during a setting time;
disassembling the counter mold 4,5 to release the mold 2 filled by the implant forming material;
optionally heating the implant by means of an external heat source;
adapting the implant by making holes or the like.

According to one embodiment, in order to avoid residual monomers, post-hardening heating is achieved preferably up to 95, 90, 85, 80 or 75° C. during 5, 10, 15, 20, 30, 45 or 60 minutes.

According to one embodiment, post heating is achieved preferably up to 90° C. during 15 minutes.

According to one embodiment, the process is exothermic. According to one embodiment, if the process is not exothermic the heat source is provided by an external source.

According to one embodiment, the implant forming material may be shaped (shaping time) between 30 seconds and 15 minutes after mixing the solid phase and the liquid phase of the implant forming material, preferably between 1 and 12 minutes, more preferably between 1 and 7 minutes. According to one embodiment, in order to extend the shaping time, the liquid and solid phases of the implant forming material are cooled down before mixing.

According to one embodiment, the setting time is ranging from 5 to 30 minutes, preferably from 10 to 15 minutes.

The invention also relates to a cranioplasty method of a subject comprising the following steps:
providing:
   a mold 2 having a surface concave along at least one axis obtained by additive manufacturing from medical imaging;
   optionally, a molding ring 3 obtained by additive manufacturing from medical imaging;
   optionally, a forming tool obtained by additive manufacturing from medical imaging;
   an implant forming material comprising a composition comprising a liquid phase comprising at least one monomer and at least one polymerization inhibitor; and a solid phase comprising at least one polymer, at least one initiator, at least one activator and at least one radio-opaque filler; and
   means for retaining the implant forming material in the mold 2 during hardening and for unmolding the hardened implant thereafter; and
coating the concave surface of the mold 2 with the means for retaining the implant forming material in the mold 2 during hardening and for unmolding the hardened implant thereafter;
mixing the liquid phase comprising at least one liquid monomer and at least one polymerization inhibitor; with the solid phase comprising at least one polymer, at least one initiator, at least one activator; and at least one radio-opaque filler;
shaping the implant forming material by spreading out the implant forming material onto the concave surface of the mold 2 and optionally by means of the molding ring 3 and/or the forming tool;
testing the implant on the patient's skull after removal of the optional molding ring 3 and the optional forming tool during the shaping time;
optionally, reshaping the implant;
hardening of the implant during a setting time;
unmolding the implant from the mold 2;
optionally heating the implant by means of an external source;
adapting the implant by making holes or the like;
placing the implant into the patient's skull; and
fixing the implant into the patient's skull by means of fixation known by one skilled in the art.

According to one embodiment, the implant forming material may be shaped during a shaping time ranging from 30 seconds to 15 minutes after mixing the solid phase and the liquid phase of the implant forming material, preferably between 1 and 12 minutes, more preferably between 1 and 7 minutes. According to one embodiment, in order to extend the shaping time, the liquid and solid phases of the implant forming material are cooled down before mixing.

According to one embodiment, the setting time is ranging from 1 to 15 minutes or from 2 to 10 minutes or from 3 to 5 minutes.

According to one embodiment, in order to avoid residual monomers, post-hardening heating is achieved preferably up to 95, 90, 85, 80 or 75° C. during 5, 10, 15, 20, 30, 45 or 60 minutes.

According to one embodiment, post heating is achieved preferably up to 90° C. during 15 minutes.

According to one embodiment, the process is exothermic. According to one embodiment, if the process is not exothermic the heat source is provided by an external source.

According to one embodiment, the implant molding system is sterilized, for instance by autoclave, γ irradiation, ethylene oxide sterilization or gas plasma sterilization. According to one embodiment, the coating resists to sterilization processes such as but not limited to EtO sterilization, γ irradiation, gas plasma sterilization; preferably is stable in autoclave.

The present invention has been described in terms of specific embodiments, which are illustrative of the invention and not to be construed as limiting. More generally, it will be appreciated by persons skilled in the art that the present invention is not limited by what has been particularly shown and/or described hereinabove.

### BRIEF DESCRIPTION OF THE DRAWINGS

The foregoing summary and detailed description of the invention will be better understood when read in conjunction with the appended drawings. For the purpose of illustrating the invention, there are shown in the drawings, certain embodiments which are presently preferred. It should be understood, however, that the invention is not limited to the precise arrangements and instrumentalities shown.

FIG. 1 illustrates a bone defect 1 to be repaired or restored.

FIGS. 2A and 2B illustrate a mold 2 according to one embodiment of the invention.

FIG. 3 illustrates a bone defect 1 to be repaired or restored together with a molding ring 3 and a mold 2 according to one embodiment of the invention. The mold 2 has the curvature of the bone defect 1 to be repaired or restored while the inner edge of the molding ring 3 has the thickness and shape of the bone defect 1 at its periphery.

FIGS. 4A and 4B illustrate a mold 2 and a molding ring 3 according to one embodiment of the invention.

FIGS. 5A and 5B illustrate a mold 2 according to one embodiment of the invention positioned in a bone defect 1 in order to test in situ the hardening implant.

FIG. 6 illustrates a counter mold 4 divided into two parts and a mold 2 with a handle 23.

FIG. 7 illustrates an assembled counter mold 4 and a mold 2 with a handle 23.

FIGS. 8A and 8B illustrate an assembled counter mold 4 and a mold 2 with a handle 23 with a view from each face.

FIGS. 9A and 9B illustrate the two portions, 41 and 42, of the counter mold 4 and mold 2 assembled wherein the cavity has the desired shape for the implant.

FIGS. 10A, 10B, 10C and 10D illustrate a counter mold 5 divided into two portions 51, 52.

The drawings are not drawn to scale and are not intended to limit the scope of the claims to the embodiments depicted.

### REFERENCES

1—Bone defect of a patient;
2—Mold;
   21—Outer face of the mold;
   22—Inner face of the mold;
   23—Handle;
   24—First mold portion;
      241—Handle;
   25—Second mold portion;
      251—Handle;
3—Molding ring;
4—Counter mold;
   41—First counter mold portion;
      411—Inner part;
         4111—Inner face;
         4112—Outer face;
      412—Outer part;
         4122—Outer face;
      413—Fixing systems;
   42—Second counter mold portion;
      421—Inner part;
         4211—Inner face;
         4212—Outer face;
      422—Outer part;
         4222—Outer face;
      423—Fixing systems;
   43—Fixing systems;
5—Counter mold;
   51—First counter mold portion;
   52—Second counter mold portion.

### EXAMPLE

The following example will be better understood when read in conjunction with the drawings.

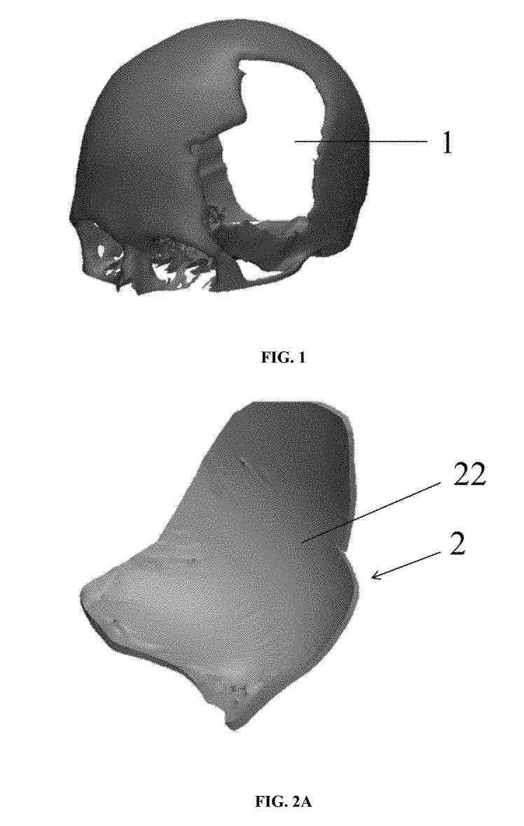

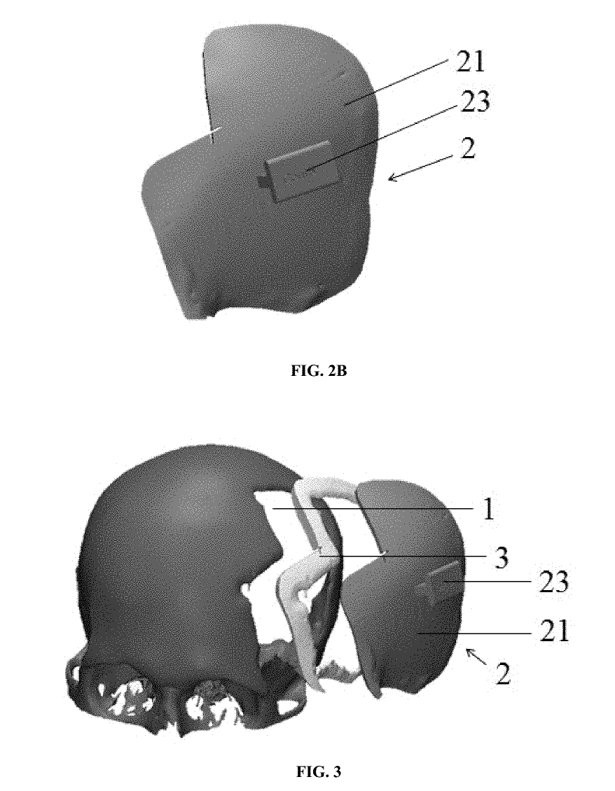

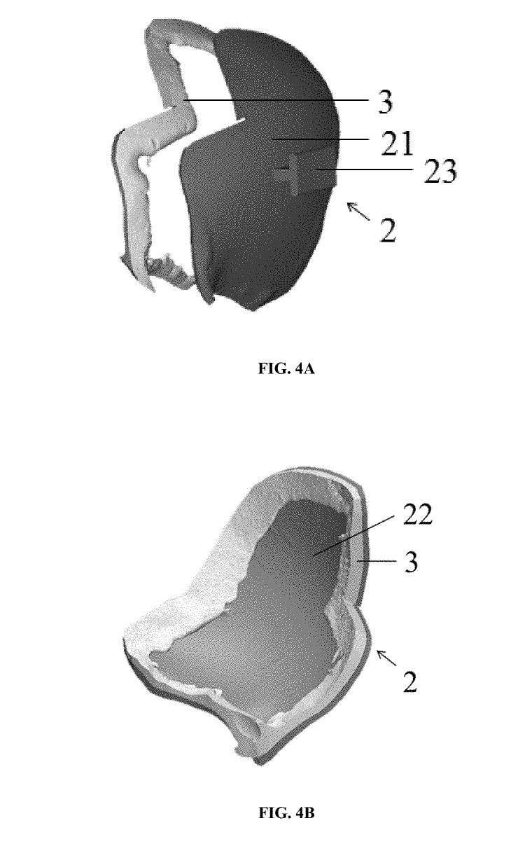

In order to confirm the ability of a PEG 1000-coating on a polyamide mold to resolve the adhesion and contamination issues, an implant has been manufactured according to the present invention using the following steps:
obtaining an image of a bone defect of a patient 1 by means of CT-scans, as represented in FIG. 1;
manufacturing a mold 2, as represented in FIGS. 2A and 2B, in polyamide (PA12) having the curvature of the bone defect 1 and a surface larger than the bone defect 1 itself by mean of 3D printing;
manufacturing a molding ring 3, as represented in FIG. 3, in polyamide (PA12) wherein the inner edge of the molding ring 3 has the thickness and shape of the bone defect 1 at its periphery by mean of 3D printing;
heating a PEG 1000 composition at 80° C. during 5 minutes in order to melt it;
impregnating a gauze with the melted PEG 1000 composition;
rubbing the impregnated gauze on the mold;
let the PEG 1000 coating hardened on the mold at room temperature during 5 minutes;
inserting/cooperating the molding ring 3 with the mold 2, as illustrated in FIGS. 4A and 4B;
shaping a Palacos® composition on the coated mold;
let the implant hardened on the mold; as the hardening reaction is exothermic, the PEG 1000 coating shall melt; and
demolding the implant from the mold.

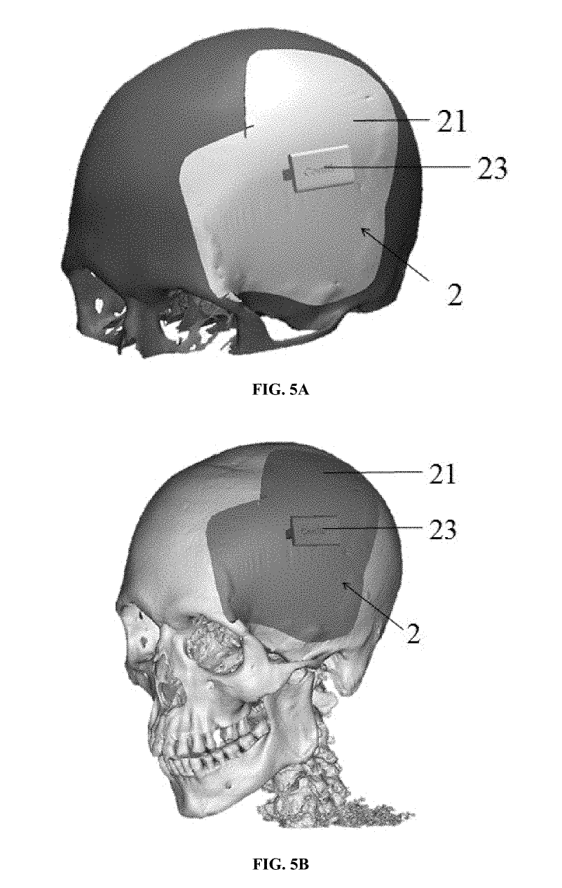

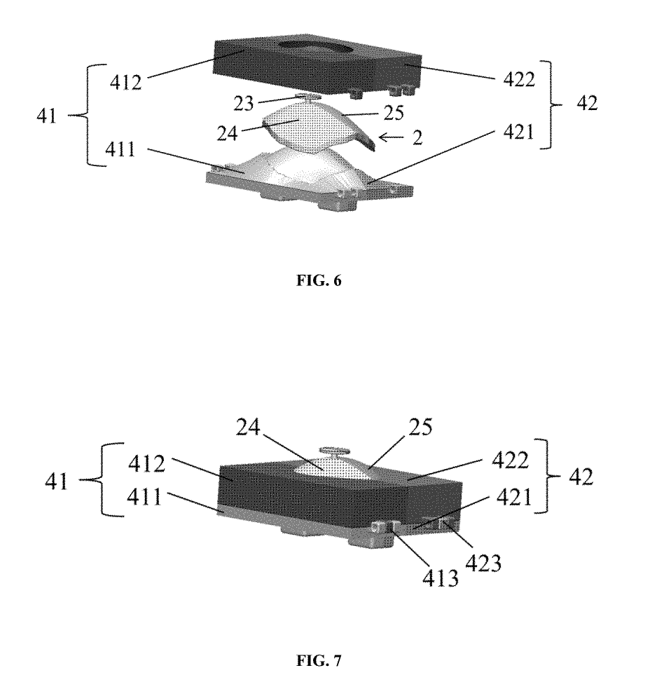

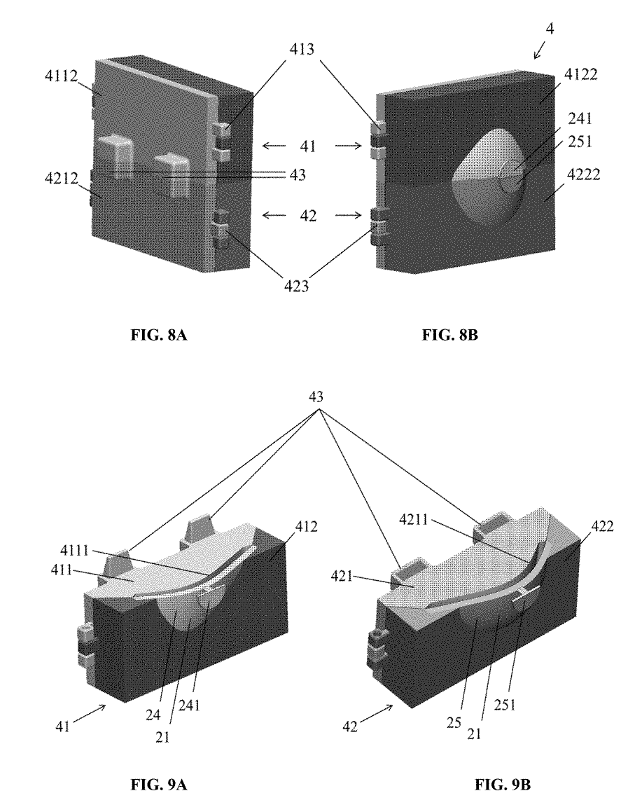

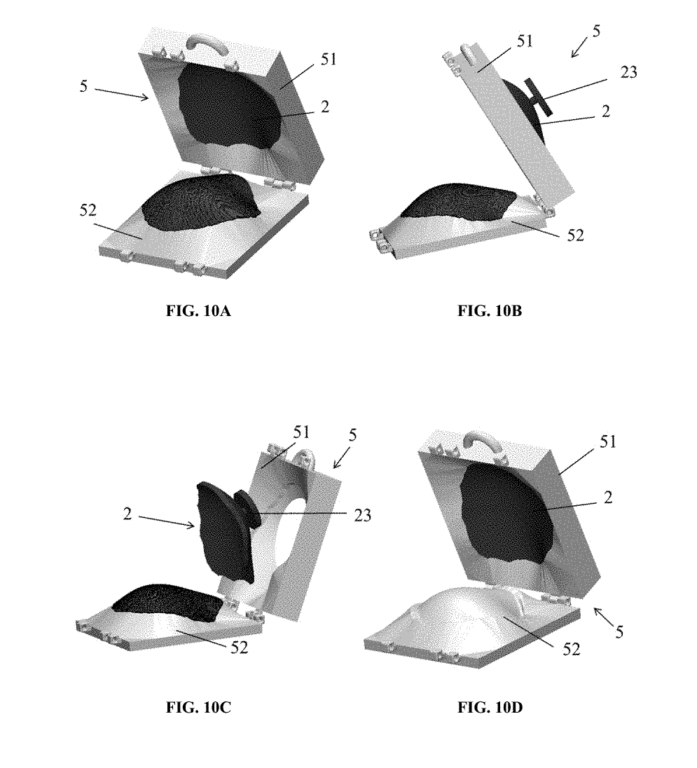

According to one embodiment, during the shaping of the Palacos® composition, the molding ring 3 may be removed from the mold 2 and the implant may be tested on the patient by means of the mold 2, as illustrated in FIGS. 5A and 5B.

1-15. (canceled)

16. An implant molding system for the molding of an hardenable implant forming material comprising:
a mold obtainable from an image of a bone defect by additive manufacturing;
said mold having a surface concave along at least one axis;
the concave surface along at least one axis conforms to an arc with a radius ranging from 2 to 20 centimeters;
the implant molding system further comprising tangible means for retaining the implant forming material, for instance against gravity, in the mold during hardening and for unmolding the implant after hardening;
said tangible means for retaining the implant forming material in the mold during hardening and for unmolding after hardening comprises or consists of a coating, said coating being applied onto the concave surface of the mold; wherein said coating is made of a biocompatible material and said coating has a phase transition temperature ranging from 40 to 100° C.

17. The implant molding system according to claim 16, wherein said coating is selected from vaseline, or Horsley's wax or homopolymer or copolymer of poly(isoprene), poly(chloroprene), poly(vinyl alcohol), poly(acrylonitrile), poly(butadiene), poly(vinyl chloride), poly(ethylene), poly(isobutylene), poly(ethylene glycol), poly(lactic acid) or any mixtures thereof.

18. The implant molding system according to claim 16, wherein said coating forms a solid layer at a temperature ranging from 0 to 30° C.

19. The implant molding system according to claim 16, wherein said coating forms a solid layer at a temperature ranging from 15 to 25° C.

20. The implant molding system according to claim 16, wherein said coating is applied onto the concave surface of the mold in solid form.

21. The implant molding system according to claim 20, wherein said coating further comprises a tab extending outside of the mold enabling peeling said coating off the mold.

22. The implant molding system according to claim 20, wherein said coating is selected from poly(acrylonitrile-co-butadiene).

23. The implant molding system according to claim 16, wherein said coating is applied onto the concave surface of the mold in liquid form.

24. The implant molding system according to claim 23, wherein the coating exhibits a melting temperature above T1 and below T2, wherein T1 is ranging from 0 to 40° C. and wherein T2 is ranging from ranging from 45 to 100° C.

25. The implant molding system according to claim 23, wherein said coating is selected from Horsley's wax or vaseline or homopolymer or copolymer of poly(ethylene glycol) or any mixtures thereof.

26. The implant molding system according to claim 16, further comprising a molding ring having a negative surface profile relative to the mold, an inner edge and a predefined thickness along the edge.

27. The implant molding system according to claim 16, further comprising a forming tool having a negative surface profile relative to the mold, such that joining the forming tool with the mold forms a cavity.

28. The implant molding system according to claim 16, further comprising a forming tool having a negative surface profile relative to the mold, such that joining the forming tool with the mold, and with the molding ring, forms a cavity.

29. The implant molding system according to claim 16, further comprising a counter mold comprising a first and a second counter mold portion, each comprising an inner part and an outer part, wherein the counter mold and the mold form, when mated together, a cavity corresponding to the image of the bone defect.

30. The implant molding system according to claim 16, wherein the surface concave along at least one axis conforms to an arc with a radius ranging from 4.5 to 9.5 centimeters, or substantially equal to 7 centimeters.

31. The implant molding system according to claim 16, wherein the mold and/or the molding ring and/or the forming tool comprises a material selected from ceramics, metals, metal alloys, glasses, polymers, bone or portland like cement, gypsum, sand or mixture thereof.

32. The implant molding system according to claim 16, further comprising an hardenable implant forming composition comprising a liquid phase comprising at least one liquid monomer, and at least one polymerization inhibitor; and a solid phase comprising at least one polymer, at least one initiator.

33. The implant molding system according to claim 16, further comprising an hardenable implant forming composition comprising a liquid phase comprising at least one liquid monomer, and at least one polymerization inhibitor; and a solid phase comprising at least one polymer, at least one initiator, at least one activator.

34. The implant molding system according to claim 16, further comprising an hardenable implant forming composition comprising a liquid phase comprising at least one liquid monomer, and at least one polymerization inhibitor; and a solid phase comprising at least one polymer, at least one initiator, at least one activator, and at least one radio-opaque filler.
